SEQUENCE LISTING (9 sequences)

SEQ ID NO: 1 - DNA, 6471 bp, Artificial Sequence
  This sequence is synthesized in lab.
tacaagatgttgcatcactttactttaattgcatgatttatcagaacaactattaacatacgaagtaccattcagttcagctgcaggtataggcagtgacaagtatctaattcttagaagaatcacttactcccacaatctgtccagacacattaatctaaggacaagtttataaatagcaaacgtgattttcacattgcagtgttctcaagaatgtatatacaagtgtgtagtcctgttgatgggatgtttccccgagttctttctattgatgcgttcatgctcttgaccctggtagagacagttctttctttccacagagcagattttcttttgtcatccaccatttacaatactctgtgaattagtattacttattacatttatgaaacggcaatatttggattgtaatagctcttcagtacaattccttgtgtcttctggtaagtctctgcactatcagagagcttttagttataatcattttcctgcaacaacagccaaactcaactattgagtttcagtgtgacacaccctctttgtagcttgctgggttaacccttggcttcaagtcctgatgatgtaatgagggtggggtgtattggcaatcagtacatttccttatcgcaatttacagtcattgaaaatcatgctgtcattaatcccagtctgacataccttttctaaaatgttcacagtgcagtgtttttgtggcctaacaaaatttttctcatatcattaaaaataaacatttttataaaaaatataacactttaaatgtttacgtcgacaaaaccagttagagtaacctacaccacatgcactatacagtagcaagcacaaaattccacagaatgaagcatcacaaagttctgctcagggtggctattccatctaggtgaaatagctgggattttcaattgcctttttcatttgtttctaaagtatgttttgcttaacataaaacacaccctaatgcaaaataaaactccccaaaagttttgtttccaattgcttgcgaggtgggaacctgccaccgagacagaggctaatcttttcaatccatccaccctttctttgctctacctatgagctgtgattggaaccaatgaaccttttagtaaaatgtatcctgctttacaaacatgctgagttatctttaaaaatatttatcaacaaattacttgtcttattttgagttttcatttaaaaaaatacacacaaaacatctacatgttcacattcattagatcagagtagcatcattctcaaacagtgggtttttatatgacaactaaatatttcatgatgactaaattatttcatacaaattttcttaatgtcatctatgtacattacatcctgttcatttataaacatgttttgtgtgatgctattttgaaactttgactgtattgcctataaatattcttccaaccaaaggaatgtctatacaaaaatttataagggctgggcgcagtggctcatgcctgtaatcctagcactttgggaggctgaggtgggtggatttcttgagtccaggagtttgaaaccagccttggcaacatggcaaaaccccgtctctacaaaaaatacaaaaattagccggggatggtggtgtgcgcctgtagtcccagctattcgggaggcttgagcctgggaggtggaggttgcagtgagctatgatcacaccactgcactacagccctggcaacagagtgagatcctgtctcaaaaaacaaacaaacatttaaaagggaatgtttacctaatgggtgaatgacaccatcagtaatgaaaaatgttattgaccatacaacacacaaaagtgttttgaaacactagaattctaaatgagtggcctgttctggggctgccgctcctgtcctgagcattactatctcttctgagttttctgtgatcaagaagccctcaatatttcctgcttcaatttcccatatgaaatcaagtggtacctgatttttaaaaaattcaacccagaattcaagttttggactccaatatgacaggagtgttgtcacggctgcaggccattggtccgtggcctgtgggaccaagcctctggttctgatgctctgtcagataagaaattccttagaatccagtaatttaaataacaatacaagtcctataatagtgcaattttggcaagagcaaagaatatcgatggccttttaaaggtcaggcagtgaaaaaaccattggacaaagtgtggactgttgctttgacatagtactagcactatgatgtctcccagaaggaggctggtcgtgtgtccacctcatcatcagcgttatcttctgatgacaacagagaaggatacggagcgacacattttacgttcacataagtagcgttcacatggacatagtgctccccaatgaaagtagagaagatcgctgatatccgggacaccagttcagaaaaggatgggcgcatttcggctttagggtgccagcattttagcattacttcatataaggggtctgggcagtattcgggttgtaggagtcttctcccttgcaacaagtaaacagttatatcaaaggtgtttacgtcaggataaggtggggctcctcttgtcatcagctcccagaggagcacgccaaaggaccaatacagtttcttgcagccaagtctctgtggacaaactttttgcttgcaagatatttcatgcctttggctacttgaagaccaaagccaataagatcttttacagttggattatgagtctcatttcgaatgaaatttcgaagatctccatgtttcatgtatggtaggaccaccagcggagacccttcacttcgcaggcagattcccaggagcgagaggacattgggatgactaaaatctttcatgatgattccctcggtcagaaattgggaaacttctcctatgtcagtgattctgttcaaggatttcacagcacagtgaattttcttgccatcattgtccaacaaagtcccatgatatacacaaccaaaatgccctcttcctatgacttcattgaaatgcacaatcaggctactgggcccaatcactacatgctgcactgcctggaccagctctggatttagagcactgaggtcaatgtggacagtattttgcagtaatggactggatatatcagagtccccactagttaggatgggggacatgtctgtcagaggatactgcacttgtcggcatgaaccgttctgagatgaattaggaaactgatcttctggaaaagtagctcggtagtctacagattcatttgaaaccatttctgtagttgggcttacacttcgggcacttacaagcctatccaaatgaggagtgtgtactcttgcatcgtagcgaactaattcactgcccagatctttaatttgctttctctttttcagccacaggaaaaacccaagtagtaataacagtgctgttgatattgagacaacaccagcaatcaatcctgtgaaattctgatctggttgaactattacttttccaaggacggttgaagaaattgcttgcttccactctatatttagctcgctgttcaatttcagcaggtcattggggaccgtgcataaaacggcttcagaatgtaagtgtatattctcacagctcttatttccaacttttaacacttcacctttaactgcttcagggtcaatatcatttcccttaatttccagtacattttcattgcccattgagatcatcactggcttttcaaaaggcttaaacacaggattatgtacataaatgagatcaaagtatttggaaaggatcccatctaacatgaaaaaggctttggttttcagggggagttgcagattcagctgttgcagggaaggagtggtacaacagattatctctgaattagagcgatgttgacatgccactgtaaagttccttcctgcttcatgcacatttatgaccattctcgggacactaactgaattcaggtttttcccaacacctgttattgtgctcccaccactaataaaagatttggttggatgaatttcatagacaatgggatcttcacggtaactgaagatgcttgtctctcggttggctaagtcaattttcaatttaacagcaaactcagttgaaatggtttgggctggggtataacattcaagaatactgtttgacacactttttaaagtacatgtttttccaccaattgaaatgtgtctagaattcccactgtttaggtaatttccagttaaagtaagtaaagtgccaccagccataggaccgtatttcggcgaaatacttgttattacaggatccacataggagaatgtactgtattgtgttgtcccgtggccatttgaaataattatggacatattgaaatgcttattcatggcaggaccaactgtgcatttcaatgtattcatcgtgctctcacttaaagtcaaggtgcagctctcatttccaaggagaactctagttttctttaaatcaaatttattattcctccgaaatccaaagtcccagccacatatggtcagccttgtccctccttcaaggggtgcactatttgggaaaaccttgtagattgcaggcagacagatctgttgagtccatgtcccgctcaggcattcctccgatcgcacacatttgtcgtggcaccagccacactgaacaaagggtggggcagagaggcattgactgcaggactggaaatgtctgcagcccaagccattcaatgggatcttcgtgatcttcttcccagtgataaccagtgtgtagccattttggtttaatgtatgctccacaatcacttctggagacactggatgggagtccaggagaaaattcacatgaggggttgatggtcctgatcgagaaaccacaacctgcatgaagcgaccctctgatgtcccaagattagctatggtgaggtctcctttaatgaaggtggatatagatgttaagaggacttcgctgaattgacccatgaataagtcaacgcgctgcaaagctgtggtaaactctgttcgatattcatcacggcgcgcttcacagcctgatgaatttctcagaagtgtcctattaaagcagtgctcatgattgggtccgtaaaaatgctggagacatctcacattgtttttgttgacgatcttgttgaagaagtcgttgacatatttgatagggaatgcacacatggcagatcgatccattggttcggcagaatctggcttgctttgtgcgaacaccccgaaaagaatgtcatcattcaggctggctcctatttgtctagcaagctgggccccaggcttgctgacatacgcagcctgaagtatattaaacacttccttctttgtggatctcttttttctcttttctgtgagaatacactccagaggcatttccatgtaggaatgcaatccagagtttatggaacagaacctgattattcttgtgtgaaaagtctgagcatctagagtttccctttggaccgtcaagaagtaaataaaattgttgctttcaaaggcatggacatacttaatggggtaagaatctctgaactcaggtaaaacatcaatgtaggactggtccgtcaaaaacataaaaccatctttcgtttcctttagccttctcactgatatcgaatgcaatggatgatctgggaaataagaagaatttatggtattgcctacaaagaagttgatgaaccggtcctttacagatgaaaggactttggctcccagggcgctcaccacacagtcaggacactggctgggctcttctatctgtggggagaatatgcagtgaacctccgactgtatgtcagcagtatgattgtggggaaagacatgtcgctggcaggtccctctgttgacgctgccacagctaatgagttgatcatcatagtaggtgtcgacaactagagccatgttgatgttatctttccaaacacctcctgataaattggctttgctgctgcagtcctgacatgggaaacaatctgggtgttccagcacaggcccagtcttgtactcagcaaccttctgaaggtcttcctcatttaaaacataaatgtagttagtggcaccaaggaaaatgtgatgctcatgtagaatgacattctggatgggtgtttccgcggtgaagttgggaagctgatacttcatattcacattcatctcggactttgctagtgcctctttacactccccattgctcctctgcaccaaggtaaacaggagcacgaggatgccaggtgcaagcacagcgggggccttcattatgagaggtttatctttcggtgcccaggaaccagtggagaagtcagcggcgcaaggaccacacgcgcgctccgcgcctccccgcctcctctcagcaagtcagctgtcgccccgcatctggctcgcgccctccactcggctccgcatctgctcacaaagcgctcggggcgccgcgggcggcgagggcctccgggtcacctgc

SEQ ID NO: 2 - RNA, 6471 nt, Artificial Sequence
  This sequence is synthesized in lab.
gcaggugacccggaggcccucgccgcccgcggcgccccgagcgcuuugugagcagaugcggagccgaguggagggcgcgagccagaugcggggcgacagcugacuugcugagaggaggcggggaggcgcggagcgcgcgugugguccuugcgccgcugacuucuccacugguuccugggcaccgaaagauaaaccucucauaaugaaggcccccgcugugcuugcaccuggcauccucgugcuccuguuuaccuuggugcagaggagcaauggggaguguaaagaggcacuagcaaaguccgagaugaaugugaauaugaaguaucagcuucccaacuucaccgcggaaacacccauccagaaugucauucuacaugagcaucacauuuuccuuggugccacuaacuacauuuauguuuuaaaugaggaagaccuucagaagguugcugaguacaagacugggccugugcuggaacacccagauuguuucccaugucaggacugcagcagcaaagccaauuuaucaggagguguuuggaaagauaacaucaacauggcucuaguugucgacaccuacuaugaugaucaacucauuagcuguggcagcgucaacagagggaccugccagcgacaugucuuuccccacaaucauacugcugacauacagucggagguucacugcauauucuccccacagauagaagagcccagccaguguccugacuguguggugagcgcccugggagccaaaguccuuucaucuguaaaggaccgguucaucaacuucuuuguaggcaauaccauaaauucuucuuauuucccagaucauccauugcauucgauaucagugagaaggcuaaaggaaacgaaagaugguuuuauguuuuugacggaccaguccuacauugauguuuuaccugaguucagagauucuuaccccauuaaguauguccaugccuuugaaagcaacaauuuuauuuacuucuugacgguccaaagggaaacucuagaugcucagacuuuucacacaagaauaaucagguucuguuccauaaacucuggauugcauuccuacauggaaaugccucuggaguguauucucacagaaaagagaaaaaagagauccacaaagaaggaaguguuuaauauacuucaggcugcguaugucagcaagccuggggcccagcuugcuagacaaauaggagccagccugaaugaugacauucuuuucgggguguucgcacaaagcaagccagauucugccgaaccaauggaucgaucugccaugugugcauucccuaucaaauaugucaacgacuucuucaacaagaucgucaacaaaaacaaugugagaugucuccagcauuuuuacggacccaaucaugagcacugcuuuaauaggacacuucugagaaauucaucaggcugugaagcgcgccgugaugaauaucgaacagaguuuaccacagcuuugcagcgcguugacuuauucaugggucaauucagcgaaguccucuuaacaucuauauccaccuucauuaaaggagaccucaccauagcuaaucuugggacaucagagggucgcuucaugcagguugugguuucucgaucaggaccaucaaccccucaugugaauuuucuccuggacucccauccagugucuccagaagugauuguggagcauacauuaaaccaaaauggcuacacacugguuaucacugggaagaagaucacgaagaucccauugaauggcuugggcugcagacauuuccaguccugcagucaaugccucucugccccacccuuuguucaguguggcuggugccacgacaaaugugugcgaucggaggaaugccugagcgggacauggacucaacagaucugucugccugcaaucuacaagguuuucccaaauagugcaccccuugaaggagggacaaggcugaccauauguggcugggacuuuggauuucggaggaauaauaaauuugauuuaaagaaaacuagaguucuccuuggaaaugagagcugcaccuugacuuuaagugagagcacgaugaauacauugaaaugcacaguugguccugccaugaauaagcauuucaauauguccauaauuauuucaaauggccacgggacaacacaauacaguacauucuccuauguggauccuguaauaacaaguauuucgccgaaauacgguccuauggcugguggcacuuuacuuacuuuaacuggaaauuaccuaaacagugggaauucuagacacauuucaauugguggaaaaacauguacuuuaaaaagugugucaaacaguauucuugaauguuauaccccagcccaaaccauuucaacugaguuugcuguuaaauugaaaauugacuuagccaaccgagagacaagcaucuucaguuaccgugaagaucccauugucuaugaaauucauccaaccaaaucuuuuauuaguggugggagcacaauaacagguguugggaaaaaccugaauucaguuagugucccgagaauggucauaaaugugcaugaagcaggaaggaacuuuacaguggcaugucaacaucgcucuaauucagagauaaucuguuguaccacuccuucccugcaacagcugaaucugcaacucccccugaaaaccaaagccuuuuucauguuagaugggauccuuuccaaauacuuugaucucauuuauguacauaauccuguguuuaagccuuuugaaaagccagugaugaucucaaugggcaaugaaaauguacuggaaauuaagggaaaugauauugacccugaagcaguuaaaggugaaguguuaaaaguuggaaauaagagcugugagaauauacacuuacauucugaagccguuuuaugcacgguccccaaugaccugcugaaauugaacagcgagcuaaauauagaguggaagcaagcaauuucuucaaccguccuuggaaaaguaauaguucaaccagaucagaauuucacaggauugauugcugguguugucucaauaucaacagcacuguuauuacuacuuggguuuuuccuguggcugaaaaagagaaagcaaauuaaagaucugggcagugaauuaguucgcuacgaugcaagaguacacacuccucauuuggauaggcuuguaagugcccgaaguguaagcccaacuacagaaaugguuucaaaugaaucuguagacuaccgagcuacuuuuccagaagaucaguuuccuaauucaucucagaacgguucaugccgacaagugcaguauccucugacagacaugucccccauccuaacuaguggggacucugauauauccaguccauuacugcaaaauacuguccacauugaccucagugcucuaaauccagagcugguccaggcagugcagcauguagugauugggcccaguagccugauugugcauuucaaugaagucauaggaagagggcauuuugguuguguauaucaugggacuuuguuggacaaugauggcaagaaaauucacugugcugugaaauccuugaacagaaucacugacauaggagaaguuucccaauuucugaccgagggaaucaucaugaaagauuuuagucaucccaauguccucucgcuccugggaaucugccugcgaagugaagggucuccgcuggugguccuaccauacaugaaacauggagaucuucgaaauuucauucgaaaugagacucauaauccaacuguaaaagaucuuauuggcuuuggucuucaaguagccaaaggcaugaaauaucuugcaagcaaaaaguuuguccacagagacuuggcugcaagaaacuguauugguccuuuggcgugcuccucugggagcugaugacaagaggagccccaccuuauccugacguaaacaccuuugauauaacuguuuacuuguugcaagggagaagacuccuacaacccgaauacugcccagaccccuuauaugaaguaaugcuaaaaugcuggcacccuaaagccgaaaugcgcccauccuuuucugaacuggugucccggauaucagcgaucuucucuacuuucauuggggagcacuauguccaugugaacgcuacuuaugugaacguaaaaugugucgcuccguauccuucucuguugucaucagaagauaacgcugaugaugagguggacacacgaccagccuccuucugggagacaucauagugcuaguacuaugucaaagcaacaguccacacuuuguccaaugguuuuuucacugccugaccuuuaaaaggccaucgauauucuuugcucuugccaaaauugcacuauuauaggacuuguauuguuauuuaaauuacuggauucuaaggaauuucuuaucugacagagcaucagaaccagaggcuuggucccacaggccacggaccaauggccugcagccgugacaacacuccugucauauuggaguccaaaacuugaauucuggguugaauuuuuuaaaaaucagguaccacuugauuucauaugggaaauugaagcaggaaauauugagggcuucuugaucacagaaaacucagaagagauaguaaugcucaggacaggagcggcagccccagaacaggccacucauuuagaauucuaguguuucaaaacacuuuuguguguuguauggucaauaacauuuuucauuacugauggugucauucacccauuagguaaacauucccuuuuaaauguuuguuuguuuuuugagacaggaucucacucuguugccagggcuguagugcaguggugugaucauagcucacugcaaccuccaccucccaggcucaagccucccgaauagcugggacuacaggcgcacaccaccauccccggcuaauuuuuguauuuuuuguagagacgggguuuugccauguugccaaggcugguuucaaacuccuggacucaagaaauccacccaccucagccucccaaagugcuaggauuacaggcaugagccacugcgcccagcccuuauaaauuuuuguauagacauuccuuugguuggaagaauauuuauaggcaauacagucaaaguuucaaaauagcaucacacaaaacauguuuauaaaugaacaggauguaauguacauagaugacauuaagaaaauuuguaugaaauaauuuagucaucaugaaauauuuaguugucauauaaaaacccacuguuugagaaugaugcuacucugaucuaaugaaugugaacauguagauguuuuguguguauuuuuuuaaaugaaaacucaaaauaagacaaguaauuuguugauaaauauuuuuaaagauaacucagcauguuuguaaagcaggauacauuuuacuaaaagguucauugguuccaaucacagcucauagguagagcaaagaaaggguggauggauugaaaagauuagccucugucucgguggcagguucccaccucgcaagcaauuggaaacaaaacuuuuggggaguuuuauuuugcauuaggguguguuuuauguuaagcaaaacauacuuuagaaacaaaugaaaaaggcaauugaaaaucccagcuauuucaccuagauggaauagccacccugagcagaacuuugugaugcuucauucuguggaauuuugugcuugcuacuguauagugcaugugguguagguuacucuaacugguuuugucgacguaaacauuuaaaguguuauauuuuuuauaaaaauguuuauuuuuaaugauaugagaaaaauuuuguuaggccacaaaaacacugcacugugaacauuuuagaaaagguaugucagacugggauuaaugacagcaugauuuucaaugacuguaaauugcgauaaggaaauguacugauugccaauacaccccacccucauuacaucaucaggacuugaagccaaggguuaacccagcaagcuacaaagagggugugucacacugaaacucaauaguugaguuuggcuguuguugcaggaaaaugauuauaacuaaaagcucucugauagugcagagacuuaccagaagacacaaggaauuguacugaagagcuauuacaauccaaauauugccguuucauaaauguaauaaguaauacuaauucacagaguauuguaaaugguggaugacaaaagaaaaucugcucuguggaaagaaagaacugucucuaccagggucaagagcaugaacgcaucaauagaaagaacucggggaaacaucccaucaacaggacuacacacuuguauauacauucuugagaacacugcaaugugaaaaucacguuugcuauuuauaaacuuguccuuagauuaaugugucuggacagauugugggaguaagugauucuucuaagaauuagauacuugucacugccuauaccugcagcugaacugaaugguacuucguauguuaauaguuguucugauaaaucaugcaauuaaaguaaagugaugcaacaucuugua

SEQ ID NO: 3 - protein, 1220 aa, Artificial Sequence
  This sequence is synthesized in lab.
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCIGPLACSSGS

SEQ ID NO: 4 - DNA, 22 bp, Artificial Sequence
  This sequence is synthesized in lab.
aatttctgaccgagggaatcat

SEQ ID NO: 5 - DNA, 24 bp, Artificial Sequence
  This sequence is synthesized in lab.
acgccaaaggaccaatacagtttc

SEQ ID NO: 6 - DNA, 24 bp, Artificial Sequence
  This sequence is synthesized in lab.
gccaaaggaccaatacagtttctt

SEQ ID NO: 7 - DNA, 43 bp, Artificial Sequence
  This sequence is synthesized in lab.
taatacgactcactataggggagcacgccaaaggaccaataca

SEQ ID NO: 8 - DNA, 25 bp, Artificial Sequence
  This sequence is synthesized in lab.
tgaaatatcttgcaagcaaaaagtt

SEQ ID NO: 9 - DNA, 24 bp, Artificial Sequence
  This sequence is synthesized in lab.
agcacgccaaaggaccaatacagt

(19) United States
(12) Patent Application Publication
(10) Pub. No.: US 2023/0193397 A1
(43) Pub. Date: Jun. 22, 2023

(54) cDNA, mRNA, PROTEIN, AND KIT AND SYSTEM FOR EVALUATING GLIOMA PROGNOSIS

(71) Applicant: Beijing Neurosurgical Institute, Beijing (CN)

(72) Inventors: Tao JIANG, Beijing (CN); Zheng ZHAO, Beijing (CN); Ruichao CHAI, Beijing (CN); Jing CHEN, Beijing (CN)

(73) Assignee: Beijing Neurosurgical Institute, Beijing (CN)

(21) Appl. No.: 17/919,118

(22) PCT Filed: Apr. 20, 2021
(86) PCT No.: PCT/CN2021/088497

§ 371 Date: Oct. 14, 2022

(30) Foreign Application Priority Data
Apr. 21, 2020 (CN) 202010318367.3

(51) Int. Cl. C12Q 1/6886; C12N 15/11; C07K 14/47; G16B 30/10

(52) U.S. Cl. CPC C12Q 1/6886; C12N 15/11; C07K 14/47; G16B 30/10; C12Q 2600/118

(57) ABSTRACT

Provided are cDNA, mRNA serving as a reverse transcription template of the cDNA, a protein coded by the mRNA, and a kit and system for evaluating a glioma prognosis. The sequence of the cDNA contains a fragment of SEQ ID NO: 1 or the sequence of the cDNA contains a complementary fragment of the fragment of SEQ ID NO: 1, wherein the fragment of SEQ ID NO: 1 at least contains the sequences of positions 2635-2652, 2623-2640 or 2627-2645 of SEQ ID NO: 1.

### CROSS-REFERENCE TO RELATED APPLICATION

This application is a national stage application under 35 U.S.C. 371 of International Application No. PCT/CN2021/088497, filed on Apr. 20, 2021 and designated the U.S., which claims priority to Chinese Patent Application No. 202010318367.3, filed on Apr. 21, 2020. The contents of each are herein incorporated by reference.

The material in the ASCII text file inno_13145_20221025_Sequence_listing.txt, created on Oct. 18, 2022, having the size of 29,255 bytes, filed for the present application via EFS-web in replacement of electronic sequence listing (inno_13145_20221013_Sequence_listing.txt; Size: 28,178 bytes; and Date of Creation: Oct. 10, 2022), is incorporated by reference in the specification for all purposes. The Sequence listing includes no new matter.

### TECHNICAL FIELD

The disclosure relates to the field of biotechnology, in particular to a cDNA related to glioma prognosis, an mRNA transcribed from the cDNA, a protein encoded by the mRNA, a kit for evaluating glioma prognosis and a system for evaluating glioma prognosis.

### BACKGROUND

Glioma is a general name of tumors derived from neuroepithelia, accounting for 40%-50% of craniocerebral tumors, and is the most common primary intracranial tumor. Gliomas are difficult to radically treat, often relapse after the operation, and have a poor prognosis. The World Health Organization grading system divides gliomas into grades I-IV according to the histological characteristics, and the prognosis of different grades of gliomas varies.

Accurately evaluating the prognosis of glioma patients has important clinical, scientific and social values. In clinical work, accurate prognosis evaluation can guide doctors to formulate personalized examination and treatment schedules for high-risk patients and help the doctors to formulate reasonable reexamination and follow-up visit plans, thus improving the quality of medical services. In scientific research, accurately assessing the risk level of patients' prognosis can provide an important basis for developing effective treatment schedules for high-risk patients, and can become an important reference for testing the effect of new treatments. From the perspective of society, accurate evaluation of the prognosis of patients can provide scientific survival expectations for patients and their families, guide the patients to comply with treatment plans, avoid excessive medical treatment, reduce family financial pressure, and help to improve the doctor-patient relationship.

At present, the evaluation of glioma prognosis is carried out mainly according to the pathological grade of glioma, but the prognosis of different patients with glioma of a same pathological grade may also vary greatly, and the evaluation of glioma prognosis is still not accurate enough only according to the pathological grade of glioma.

### SUMMARY

The disclosure aims to provide a cDNA related to glioma prognosis, an mRNA transcribed from the cDNA, a protein encoded by the mRNA, a kit for evaluating glioma prognosis and a system for evaluating glioma prognosis, capable of further improving the accuracy of glioma prognosis evaluation.

In a first aspect, the disclosure provides a cDNA, where a sequence of the cDNA contains a fragment of SEQ ID NO: 1, or the sequence of the cDNA contains a complementary fragment of the fragment of SEQ ID NO: 1; wherein the fragment of SEQ ID NO: 1 at least contains a sequence at positions 2635-2652, 2623-2640 or 2627-2645 of SEQ ID NO: 1.

Preferably, the fragment of SEQ ID NO: 1 at least contains a sequence at positions 2588-2687 of SEQ ID NO: 1.

Optionally, the cDNA corresponds to a human MET gene, and the sequence of the cDNA does not contain an exon 19 of the human MET gene.

Optionally, the cDNA is single-stranded DNA, and the sequence of the cDNA is the sequence as shown in SEQ ID NO: 1, or the sequence of the cDNA is a complementary sequence of the sequence as shown in SEQ ID NO: 1.

Optionally, the cDNA is double-stranded DNA, and the sequence of one single strand of the cDNA is the sequence as shown in SEQ ID NO: 1, and the sequence of the other single strand of the cDNA is the complementary sequence of the sequence as shown in SEQ ID NO: 1.

In a second aspect, the disclosure provides an mRNA, where the mRNA is a reverse transcription template for any cDNA in the first aspect.

Optionally, the sequence of the mRNA contains a fragment of SEQ ID NO: 2, and the fragment of SEQ ID NO: 2 at least contains a sequence at positions 3820-3837, 3832-3849 or 3827-3845 of SEQ ID NO: 2.

Preferably, the fragment of SEQ ID NO: 2 at least contains a sequence at positions 3785-3884 of SEQ ID NO: 2.

Optionally, the sequence of the mRNA is the sequence as shown in SEQ ID NO: 2.

In a third aspect, the disclosure provides a protein, where the protein is encoded by any mRNA in the second aspect.

Optionally, the amino acid sequence of the protein contains a fragment of SEQ ID NO: 3, and the fragment of SEQ ID NO: 3 at least contains a sequence at positions 1207-1220 of SEQ ID NO: 3.

Preferably, the fragment of SEQ ID NO: 3 at least contains a sequence at positions 1175-1220 of SEQ ID NO: 3.

Optionally, the amino acid sequence of the protein is the sequence as shown in SEQ ID NO: 3.

In a fourth aspect, the disclosure provides a kit for evaluating glioma prognosis, where the kit includes a first primer pair capable of specifically amplifying any cDNA in the first aspect, and/or a first probe capable of specifically hybridizing with any cDNA in the first aspect; and/or,

the kit includes a second primer pair capable of specifically amplifying any mRNA in the second aspect, and/or a second probe capable of specifically hybridizing with any mRNA in the second aspect, and/or,

the kit includes an antibody for resisting any protein in the third aspect, and any protein in the third aspect.

Optionally, the first primer pair contains a first primer as shown in SEQ ID NO: 4 and a second primer as shown in SEQ ID NO: 5, and the sequence of the first probe contains a sequence as shown in SEQ ID NO: 6.

The second primer pair contains a third primer as shown in SEQ ID NO: 7 and a fourth primer as shown in SEQ ID NO: 8, and the sequence of the second probe contains a sequence as shown in SEQ ID NO: 9.

In a fifth aspect, the disclosure provides application of a molecular reagent in preparation of a kit for evaluating glioma prognosis, where the molecular reagent includes at least one of (1)-(8) as follows:

(1) any cDNA in the first aspect;

(2) a first primer pair capable of specifically amplifying any cDNA in the first aspect;

(3) a first probe capable of specifically hybridizing with any cDNA in the first aspect;

(4) any mRNA in the second aspect;

(5) a second primer pair capable of specifically amplifying any mRNA in the second aspect;

(6) a second probe capable of specifically hybridizing with any mRNA in the second aspect;

(7) any protein in the third aspect; and

(8) an antibody for resisting any protein in the third aspect.

In a sixth aspect, the disclosure provides a system for evaluating glioma prognosis, where the system includes an amplification device, a sequencing device, a computing device and an output device.

The amplification device includes a collection unit and an amplification unit, where the collection unit is used for collecting a template nucleic acid fragment and an amplification primer, and the amplification unit is used for amplifying the template nucleic acid fragment by using the amplification primer to obtain an amplification product.

The sequencing device is used for sequencing the nucleic acid sequence of the amplification product to obtain a sequence of the amplification product.

The computing device includes a memory and a processor, where a computer program is stored in the memory, and the processor is configured to execute the computer program stored in the memory so as to achieve the following determination:

if the sequence of the amplification product contains the sequence of any cDNA in the first aspect and/or the sequence of any mRNA in the second aspect, it is determined that the prognosis of glioma corresponding to the template nucleic acid fragment is poor.

The output device is used for outputting a determination result of the computing device.

In a seventh aspect, the disclosure provides a method for evaluating glioma prognosis, where the method includes a step of detecting whether a to-be-detected glioma sample contains the cDNA and/or the mRNA and/or the protein as described above, and if the to-be-detected glioma sample contains the cDNA and/or the mRNA and/or the protein as described above, it is indicated that the prognosis of a glioma patient corresponding to the to-be-detected glioma sample is poorer.

Through the above technical solutions, the accuracy of glioma prognosis evaluation is effectively improved in the disclosure.

Other features and advantages of the disclosure will be described in detail in the subsequent specific examples.

### BRIEF DESCRIPTION OF THE DRAWINGS

The drawings are used to provide a further understanding of the disclosure and constitute a part of the description, and are used to explain the disclosure together with the following specific examples, but do not constitute limitations of the disclosure. In the drawings:

FIG. 1 is an agarose gel nucleic acid electrophoretogram of cDNA provided by the examples of the disclosure.

FIG. 2 is a diagram showing Sanger sequencing results of cDNA provided by the examples of the disclosure.

FIG. 3 is a survival curve graph of patients with a full-grade glioma sample provided by the examples of the disclosure.

FIG. 4 is a survival curve graph of patients with an IDH mutated full-grade glioma sample provided by the examples of the disclosure.

FIG. 5 is a survival curve graph of patients with a secondary glioblastoma sample provided by the examples of the disclosure.

FIG. 6 is a survival curve graph of patients with an IDH mutated secondary glioblastoma sample provided by the examples of the disclosure.

### DETAILED DESCRIPTION

The specific examples of the disclosure are described in detail below in combination with the drawings. It should be understood that the specific examples described herein are only used to illustrate and interpret the disclosure, but not to limit the disclosure.

In a first aspect, the disclosure provides a cDNA, where a sequence of the cDNA contains a fragment of SEQ ID NO: 1, or the sequence of the cDNA contains a complementary fragment of the fragment of SEQ ID NO: 1; wherein the fragment of SEQ ID NO: 1 at least contains a sequence at positions 2635-2652, 2623-2640 or 2627-2645 of SEQ ID NO: 1; preferably, the fragment of SEQ ID NO: 1 at least contains a sequence at positions 2588-2687 of SEQ ID NO: 1.

Preferably, the cDNA corresponds to a human MET gene, and the sequence of the cDNA does not contain an exon 19 of the human MET gene.

According to the disclosure, the cDNA can be single-stranded DNA, and the sequence of the cDNA can be the sequence as shown in SEQ ID NO: 1, or the sequence of the cDNA can be a complementary sequence of the sequence as shown in SEQ ID NO: 1.

According to the disclosure, the cDNA can be double-stranded DNA, and the sequence of one single strand of the cDNA can be the sequence as shown in SEQ ID NO: 1, and the sequence of the other single strand of the cDNA can be the complementary sequence of the sequence as shown in SEQ ID NO: 1.

The cDNA is cDNA corresponding to the human MET gene with the exon 19 deleted. The inventors of the disclosure have found that when the cDNA can be reversely transcribed from mRNA of glioma cells, the malignant degree of glioma is higher, the drug resistance is stronger, and the prognosis of patients is poorer. Where the fragment of SEQ ID NO: 1 contains a deletion site of the exon 19, the sequence shown in SEQ ID NO: 1 contains all exons, except the exon 19, of the human MET gene, and the sequence shown in SEQ ID NO: 1 is as follows:

tacaagatgttgcatcactttactttaattgcatgatttatcagaacaactattaacatacgaagtaccattcagttcagctg
caggtataggcagtgacaagtatctaattcttagaagaatcacttactcccacaatctgtccagacacattaatctaaggaca
agtttataaatagcaaacgtgattttcacattgcagtgttctcaagaatgtatatacaagtgtgtagtcctgttgatgggatg
tttccccgagttctttctattgatgcgttcatgctcttgaccctggtagagacagttctttctttccacagagcagattttct
tttgtcatccaccatttacaatactctgtgaattagtattacttattacatttatgaaacggcaatatttggattgtaatagc
tcttcagtacaattccttgtgtcttctggtaagtctctgcactatcagagagcttttagttataatcattttcctgcaacaac
agccaaactcaactattgagtttcagtgtgacacaccctctttgtagcttgctgggttaacccttggcttcaagtcctgatga
tgtaatgagggtggggtgtattggcaatcagtacatttccttatcgcaatttacagtcattgaaaatcatgctgtcattaatc
ccagtctgacataccttttctaaaatgttcacagtgcagtgtttttgtggcctaacaaaatttttctcatatcattaaaaata
aacatttttataaaaaatataacactttaaatgtttacgtcgacaaaaccagttagagtaacctacaccacatgcactataca
gtagcaagcacaaaattccacagaatgaagcatcacaaagttctgctcagggtggctattccatctaggtgaaatagctggga
ttttcaattgcctttttcatttgtttctaaagtatgttttgcttaacataaaacacaccctaatgcaaaataaaactccccaa
aagttttgtttccaattgcttgcgaggtgggaacctgccaccgagacagaggctaatcttttcaatccatccaccctttcttt
gctctacctatgagctgtgattggaaccaatgaaccttttagtaaaatgtatcctgctttacaaacatgctgagttatcttta
aaaatatttatcaacaaattacttgtcttattttgagttttcatttaaaaaaatacacacaaaacatctacatgttcacattc
attagatcagagtagcatcattctcaaacagtgggtttttatatgacaactaaatatttcatgatgactaaattatttcatac
aaattttcttaatgtcatctatgtacattacatcctgttcatttataaacatgttttgtgtgatgctattttgaaactttgac
tgtattgcctataaatattcttccaaccaaaggaatgtctatacaaaaatttataagggctgggcgcagtggctcatgcctgt
aatcctagcactttgggaggctgaggtgggtggatttcttgagtccaggagtttgaaaccagccttggcaacatggcaaaacc
ccgtctctacaaaaaatacaaaaattagccggggatggtggtgtgcgcctgtagtcccagctattcgggaggcttgagcctgg
gaggtggaggttgcagtgagctatgatcacaccactgcactacagccctggcaacagagtgagatcctgtctcaaaaaacaaa
caaacatttaaaagggaatgtttacctaatgggtgaatgacaccatcagtaatgaaaaatgttattgaccatacaacacacaa
aagtgttttgaaacactagaattctaaatgagtggcctgttctggggctgccgctcctgtcctgagcattactatctcttctg
agttttctgtgatcaagaagccctcaatatttcctgcttcaatttcccatatgaaatcaagtggtacctgatttttaaaaaat
tcaacccagaattcaagttttggactccaatatgacaggagtgttgtcacggctgcaggccattggtccgtggcctgtgggac
caagcctctggttctgatgctctgtcagataagaaattccttagaatccagtaatttaaataacaatacaagtcctataatag
tgcaattttggcaagagcaaagaatatcgatggccttttaaaggtcaggcagtgaaaaaaccattggacaaagtgtggactgt
tgctttgacatagtactagcactatgatgtctcccagaaggaggctggtcgtgtgtccacctcatcatcagcgttatcttctg
atgacaacagagaaggatacggagcgacacattttacgttcacataagtagcgttcacatggacatagtgctccccaatgaaa
gtagagaagatcgctgatatccgggacaccagttcagaaaaggatgggcgcatttcggctttagggtgccagcattttagcat
tacttcatataaggggtctgggcagtattcgggttgtaggagtcttctcccttgcaacaagtaaacagttatatcaaaggtgt
ttacgtcaggataaggtggggctcctcttgtcatcagctcccagaggagcacgccaaaggaccaatacagtttcttgcagcca
agtctctgtggacaaactttttgcttgcaagatatttcatgcctttggctacttgaagaccaaagccaataagatcttttaca
gttggattatgagtctcatttcgaatgaaatttcgaagatctccatgtttcatgtatggtaggaccaccagcggagacccttc
acttcgcaggcagattcccaggagcgagaggacattgggatgactaaaatctttcatgatgattccctcggtcagaaattggg
aaacttctcctatgtcagtgattctgttcaaggatttcacagcacagtgaattttcttgccatcattgtccaacaaagtccca
tgatatacacaaccaaaatgccctcttcctatgacttcattgaaatgcacaatcaggctactgggcccaatcactacatgctg
cactgcctggaccagctctggatttagagcactgaggtcaatgtggacagtattttgcagtaatggactggatatatcagagt
ccccactagttaggatgggggacatgtctgtcagaggatactgcacttgtcggcatgaaccgttctgagatgaattaggaaac
tgatcttctggaaaagtagctcggtagtctacagattcatttgaaaccatttctgtagttgggcttacacttcgggcacttac
aagcctatccaaatgaggagtgtgtactcttgcatcgtagcgaactaattcactgcccagatctttaatttgctttctctttt
tcagccacaggaaaaacccaagtagtaataacagtgctgttgatattgagacaacaccagcaatcaatcctgtgaaattctga
tctggttgaactattacttttccaaggacggttgaagaaattgcttgcttccactctatatttagctcgctgttcaatttcag
caggtcattggggaccgtgcataaaacggcttcagaatgtaagtgtatattctcacagctcttatttccaacttttaacactt
cacctttaactgcttcagggtcaatatcatttcccttaatttccagtacattttcattgcccattgagatcatcactggcttt
tcaaaaggcttaaacacaggattatgtacataaatgagatcaaagtatttggaaaggatcccatctaacatgaaaaaggcttt
ggttttcagggggagttgcagattcagctgttgcagggaaggagtggtacaacagattatctctgaattagagcgatgttgac
atgccactgtaaagttccttcctgcttcatgcacatttatgaccattctcgggacactaactgaattcaggtttttcccaaca
cctgttattgtgctcccaccactaataaaagatttggttggatgaatttcatagacaatgggatcttcacggtaactgaagat
gcttgtctctcggttggctaagtcaattttcaatttaacagcaaactcagttgaaatggtttgggctggggtataacattcaa
gaatactgtttgacacactttttaaagtacatgtttttccaccaattgaaatgtgtctagaattcccactgtttaggtaattt
ccagttaaagtaagtaaagtgccaccagccataggaccgtatttcggcgaaatacttgttattacaggatccacataggagaa
tgtactgtattgtgttgtcccgtggccatttgaaataattatggacatattgaaatgcttattcatggcaggaccaactgtgc
atttcaatgtattcatcgtgctctcacttaaagtcaaggtgcagctctcatttccaaggagaactctagttttctttaaatca
aatttattattcctccgaaatccaaagtcccagccacatatggtcagccttgtccctccttcaaggggtgcactatttgggaa
aaccttgtagattgcaggcagacagatctgttgagtccatgtcccgctcaggcattcctccgatcgcacacatttgtcgtggc
accagccacactgaacaaagggtggggcagagaggcattgactgcaggactggaaatgtctgcagcccaagccattcaatggg
atcttcgtgatcttcttcccagtgataaccagtgtgtagccattttggtttaatgtatgctccacaatcacttctggagacac
tggatgggagtccaggagaaaattcacatgaggggttgatggtcctgatcgagaaaccacaacctgcatgaagcgaccctctg
atgtcccaagattagctatggtgaggtctcctttaatgaaggtggatatagatgttaagaggacttcgctgaattgacccatg
aataagtcaacgcgctgcaaagctgtggtaaactctgttcgatattcatcacggcgcgcttcacagcctgatgaatttctcag
aagtgtcctattaaagcagtgctcatgattgggtccgtaaaaatgctggagacatctcacattgtttttgttgacgatcttgt
tgaagaagtcgttgacatatttgatagggaatgcacacatggcagatcgatccattggttcggcagaatctggcttgctttgt
gcgaacaccccgaaaagaatgtcatcattcaggctggctcctatttgtctagcaagctgggccccaggcttgctgacatacgc
agcctgaagtatattaaacacttccttctttgtggatctcttttttctcttttctgtgagaatacactccagaggcatttcca
tgtaggaatgcaatccagagtttatggaacagaacctgattattcttgtgtgaaaagtctgagcatctagagtttccctttgg
accgtcaagaagtaaataaaattgttgctttcaaaggcatggacatacttaatggggtaagaatctctgaactcaggtaaaac
atcaatgtaggactggtccgtcaaaaacataaaaccatctttcgtttcctttagccttctcactgatatcgaatgcaatggat
gatctgggaaataagaagaatttatggtattgcctacaaagaagttgatgaaccggtcctttacagatgaaaggactttggct
cccagggcgctcaccacacagtcaggacactggctgggctcttctatctgtggggagaatatgcagtgaacctccgactgtat
gtcagcagtatgattgtggggaaagacatgtcgctggcaggtccctctgttgacgctgccacagctaatgagttgatcatcat
agtaggtgtcgacaactagagccatgttgatgttatctttccaaacacctcctgataaattggctttgctgctgcagtcctga
catgggaaacaatctgggtgttccagcacaggcccagtcttgtactcagcaaccttctgaaggtcttcctcatttaaaacata
aatgtagttagtggcaccaaggaaaatgtgatgctcatgtagaatgacattctggatgggtgtttccgcggtgaagttgggaa
gctgatacttcatattcacattcatctcggactttgctagtgcctctttacactccccattgctcctctgcaccaaggtaaac
aggagcacgaggatgccaggtgcaagcacagcgggggccttcattatgagaggtttatctttcggtgcccaggaaccagtgga
gaagtcagcggcgcaaggaccacacgcgcgctccgcgcctccccgcctcctctcagcaagtcagctgtcgccccgcatctggc
tcgcgccctccactcggctccgcatctgctcacaaagcgctcggggcgccgcgggcggcgagggcctccgggtcacctgc.

Where the sequence at the positions 2635-2652 of SEQ ID NO: 1 is ccaatacagtttcttgca; the sequence at the positions 2623-2640 of SEQ ID NO: 1 is cacgccaaaggaccaata; and the sequence at the positions 2627-2645 of SEQ ID NO: 1 is ccaaaggaccaatacagtt.

In a second aspect, the disclosure provides an mRNA, where the mRNA is a reverse transcription template for any cDNA in the first aspect.

Preferably, the sequence of the mRNA contains a fragment of SEQ ID NO: 2, and the fragment of SEQ ID NO: 2 at least contains a sequence at positions 3820-3837, 3832-3849 or 3827-3845 of SEQ ID NO: 2; preferably, the fragment of SEQ ID NO: 2 at least contains a sequence at positions 3785-3884 of SEQ ID NO: 2.

Preferably, the sequence of the mRNA is a sequence as shown in SEQ ID NO: 2.

Where the sequence as shown in SEQ ID NO: 2 is an RNA sequence complementary to the sequence as shown in SEQ ID NO: 1, and the sequence as shown in SEQ ID NO: 2 is as follows:

gcaggugacccggaggcccucgccgcccgcggcgccccgagcgcuuugugagcagaugcggagccgaguggag
ggcgcgagccagaugcggggcgacagcugacuugcugagaggaggcggggaggcgcggagcgcgcgugugguccuugcgcc
gcugacuucuccacugguuccugggcaccgaaagauaaaccucucauaaugaaggcccccgcugugcuugcaccuggcauc
cucgugcuccuguuuaccuuggugcagaggagcaauggggaguguaaagaggcacuagcaaaguccgagaugaaugugaau
augaaguaucagcuucccaacuucaccgcggaaacacccauccagaaugucauucuacaugagcaucacauuuuccuuggu
gccacuaacuacauuuauguuuuaaaugaggaagaccuucagaagguugcugaguacaagacugggccugugcuggaacac
ccagauuguuucccaugucaggacugcagcagcaaagccaauuuaucaggagguguuuggaaagauaacaucaacauggcu
cuaguugucgacaccuacuaugaugaucaacucauuagcuguggcagcgucaacagagggaccugccagcgacaugucuuu
ccccacaaucauacugcugacauacagucggagguucacugcauauucuccccacagauagaagagcccagccaguguccu
gacuguguggugagcgcccugggagccaaaguccuuucaucuguaaaggaccgguucaucaacuucuuuguaggcaauacc
auaaauucuucuuauuucccagaucauccauugcauucgauaucagugagaaggcuaaaggaaacgaaagaugguuuuaug
uuuuugacggaccaguccuacauugauguuuuaccugaguucagagauucuuaccccauuaaguauguccaugccuuugaa
agcaacaauuuuauuuacuucuugacgguccaaagggaaacucuagaugcucagacuuuucacacaagaauaaucagguuc
uguuccauaaacucuggauugcauuccuacauggaaaugccucuggaguguauucucacagaaaagagaaaaaagagaucc
acaaagaaggaaguguuuaauauacuucaggcugcguaugucagcaagccuggggcccagcuugcuagacaaauaggagcc
agccugaaugaugacauucuuuucgggguguucgcacaaagcaagccagauucugccgaaccaauggaucgaucugccaug
ugugcauucccuaucaaauaugucaacgacuucuucaacaagaucgucaacaaaaacaaugugagaugucuccagcauuuu
uacggacccaaucaugagcacugcuuuaauaggacacuucugagaaauucaucaggcugugaagcgcgccgugaugaauau
cgaacagaguuuaccacagcuuugcagcgcguugacuuauucaugggucaauucagcgaaguccucuuaacaucuauaucc
accuucauuaaaggagaccucaccauagcuaaucuugggacaucagagggucgcuucaugcagguugugguuucucgauca
ggaccaucaaccccucaugugaauuuucuccuggacucccauccagugucuccagaagugauuguggagcauacauuaaac
caaaauggcuacacacugguuaucacugggaagaagaucacgaagaucccauugaauggcuugggcugcagacauuuccag
uccugcagucaaugccucucugccccacccuuuguucaguguggcuggugccacgacaaaugugugcgaucggaggaaugc
cugagcgggacauggacucaacagaucugucugccugcaaucuacaagguuuucccaaauagugcaccccuugaaggaggg
acaaggcugaccauauguggcugggacuuuggauuucggaggaauaauaaauuugauuuaaagaaaacuagaguucuccuu
ggaaaugagagcugcaccuugacuuuaagugagagcacgaugaauacauugaaaugcacaguugguccugccaugaauaag
cauuucaauauguccauaauuauuucaaauggccacgggacaacacaauacaguacauucuccuauguggauccuguaaua
acaaguauuucgccgaaauacgguccuauggcugguggcacuuuacuuacuuuaacuggaaauuaccuaaacagugggaau
ucuagacacauuucaauugguggaaaaacauguacuuuaaaaagugugucaaacaguauucuugaauguuauaccccagcc
caaaccauuucaacugaguuugcuguuaaauugaaaauugacuuagccaaccgagagacaagcaucuucaguuaccgugaa
gaucccauugucuaugaaauucauccaaccaaaucuuuuauuaguggugggagcacaauaacagguguugggaaaaaccug
aauucaguuagugucccgagaauggucauaaaugugcaugaagcaggaaggaacuuuacaguggcaugucaacaucgcucu
aauucagagauaaucuguuguaccacuccuucccugcaacagcugaaucugcaacucccccugaaaaccaaagccuuuuuc
auguuagaugggauccuuuccaaauacuuugaucucauuuauguacauaauccuguguuuaagccuuuugaaaagccagug
augaucucaaugggcaaugaaaauguacuggaaauuaagggaaaugauauugacccugaagcaguuaaaggugaaguguua
aaaguuggaaauaagagcugugagaauauacacuuacauucugaagccguuuuaugcacgguccccaaugaccugcugaaa
uugaacagcgagcuaaauauagaguggaagcaagcaauuucuucaaccguccuuggaaaaguaauaguucaaccagaucag
aauuucacaggauugauugcugguguugucucaauaucaacagcacuguuauuacuacuuggguuuuuccuguggcugaaa
aagagaaagcaaauuaaagaucugggcagugaauuaguucgcuacgaugcaagaguacacacuccucauuuggauaggcuu
guaagugcccgaaguguaagcccaacuacagaaaugguuucaaaugaaucuguagacuaccgagcuacuuuuccagaagau
caguuuccuaauucaucucagaacgguucaugccgacaagugcaguauccucugacagacaugucccccauccuaacuagu
ggggacucugauauauccaguccauuacugcaaaauacuguccacauugaccucagugcucuaaauccagagcugguccag
gcagugcagcauguagugauugggcccaguagccugauugugcauuucaaugaagucauaggaagagggcauuuugguugu
guauaucaugggacuuuguuggacaaugauggcaagaaaauucacugugcugugaaauccuugaacagaaucacugacaua
ggagaaguuucccaauuucugaccgagggaaucaucaugaaagauuuuagucaucccaauguccucucgcuccugggaauc
ugccugcgaagugaagggucuccgcuggugguccuaccauacaugaaacauggagaucuucgaaauuucauucgaaaugag
acucauaauccaacuguaaaagaucuuauuggcuuuggucuucaaguagccaaaggcaugaaauaucuugcaagcaaaaag
uuuguccacagagacuuggcugcaagaaacuguauugguccuuuggcgugcuccucugggagcugaugacaagaggagccc
caccuuauccugacguaaacaccuuugauauaacuguuuacuuguugcaagggagaagacuccuacaacccgaauacugcc
cagaccccuuauaugaaguaaugcuaaaaugcuggcacccuaaagccgaaaugcgcccauccuuuucugaacugguguccc
ggauaucagcgaucuucucuacuuucauuggggagcacuauguccaugugaacgcuacuuaugugaacguaaaaugugucg
cuccguauccuucucuguugucaucagaagauaacgcugaugaugagguggacacacgaccagccuccuucugggagacau
cauagugcuaguacuaugucaaagcaacaguccacacuuuguccaaugguuuuuucacugccugaccuuuaaaaggccauc
gauauucuuugcucuugccaaaauugcacuauuauaggacuuguauuguuauuuaaauuacuggauucuaaggaauuucuu
aucugacagagcaucagaaccagaggcuuggucccacaggccacggaccaauggccugcagccgugacaacacuccuguca
uauuggaguccaaaacuugaauucuggguugaauuuuuuaaaaaucagguaccacuugauuucauaugggaaauugaagca
ggaaauauugagggcuucuugaucacagaaaacucagaagagauaguaaugcucaggacaggagcggcagccccagaacag
gccacucauuuagaauucuaguguuucaaaacacuuuuguguguuguauggucaauaacauuuuucauuacugaugguguc
auucacccauuagguaaacauucccuuuuaaauguuuguuuguuuuuugagacaggaucucacucuguugccagggcugua
gugcaguggugugaucauagcucacugcaaccuccaccucccaggcucaagccucccgaauagcugggacuacaggcgcac
accaccauccccggcuaauuuuuguauuuuuuguagagacgggguuuugccauguugccaaggcugguuucaaacuccugg
acucaagaaauccacccaccucagccucccaaagugcuaggauuacaggcaugagccacugcgcccagcccuuauaaauuu
uuguauagacauuccuuugguuggaagaauauuuauaggcaauacagucaaaguuucaaaauagcaucacacaaaacaugu
uuauaaaugaacaggauguaauguacauagaugacauuaagaaaauuuguaugaaauaauuuagucaucaugaaauauuua
guugucauauaaaaacccacuguuugagaaugaugcuacucugaucuaaugaaugugaacauguagauguuuuguguguau
uuuuuuaaaugaaaacucaaaauaagacaaguaauuuguugauaaauauuuuuaaagauaacucagcauguuuguaaagca
ggauacauuuuacuaaaagguucauugguuccaaucacagcucauagguagagcaaagaaaggguggauggauugaaaaga
uuagccucugucucgguggcagguucccaccucgcaagcaauuggaaacaaaacuuuuggggaguuuuauuuugcauuagg
guguguuuuauguuaagcaaaacauacuuuagaaacaaaugaaaaaggcaauugaaaaucccagcuauuucaccuagaugg
aauagccacccugagcagaacuuugugaugcuucauucuguggaauuuugugcuugcuacuguauagugcaugugguguag
guuacucuaacugguuuugucgacguaaacauuuaaaguguuauauuuuuuauaaaaauguuuauuuuuaaugauaugaga
aaaauuuuguuaggccacaaaaacacugcacugugaacauuuuagaaaagguaugucagacugggauuaaugacagcauga
uuuucaaugacuguaaauugcgauaaggaaauguacugauugccaauacaccccacccucauuacaucaucaggacuugaa
gccaaggguuaacccagcaagcuacaaagagggugugucacacugaaacucaauaguugaguuuggcuguuguugcaggaa
aaugauuauaacuaaaagcucucugauagugcagagacuuaccagaagacacaaggaauuguacugaagagcuauuacaau
ccaaauauugccguuucauaaauguaauaaguaauacuaauucacagaguauuguaaaugguggaugacaaaagaaaaucu
gcucuguggaaagaaagaacugucucuaccagggucaagagcaugaacgcaucaauagaaagaacucggggaaacauccca
ucaacaggacuacacacuuguauauacauucuugagaacacugcaaugugaaaaucacguuugcuauuuauaaacuugucc
uuagauuaaugugucuggacagauugugggaguaagugauucuucuaagaauuagauacuugucacugccuauaccugcag
cugaacugaaugguacuucguauguuaauaguuguucugauaaaucaugcaauuaaaguaaagugaugcaacaucuugua.

Where the sequence at the positions 3820-3837 of SEQ ID NO: 2 is ugcaagaaacuguauugg; the sequence at the positions 3832-3849 of SEQ ID NO: 2 is uauugguccuuuggcgug; and the sequence at the positions 3827-3845 of SEQ ID NO: 2 is aacuguauugguccuuugg.

In a third aspect, the disclosure provides a protein, where the protein is encoded by any mRNA in the second aspect.

Preferably, the amino acid sequence of the protein contains a fragment of SEQ ID NO: 3, and the fragment of SEQ ID NO: 3 at least contains a sequence at positions 1207-1220 of SEQ ID NO: 3.

Preferably, the fragment of SEQ ID NO: 3 at least contains a sequence at positions 1175-1220 of SEQ ID NO: 3.

Preferably, the amino acid sequence of the protein is the sequence as shown in SEQ ID NO: 3.

Where the sequence as shown in SEQ ID NO: 3 is an amino acid sequence encoded by the RNA sequence as shown in SEQ ID NO: 2, and the amino acid sequence as shown in SEQ ID NO: 3 is as follows:

MKAPAVLAPGILVLLFTLVQRSNGECKEALAKSEMNVNMKYQLPNFTAET
PIQNVILHEHHIFLGATNYIYVLNEEDLQKVAEYKTGPVLEHPDCFPCQD
CSSKANLSGGVWKDNINMALVVDTYYDDQLISCGSVNRGTCQRHVFPHNH
TADIQSEVHCIFSPQIEEPSQCPDCVVSALGAKVLSSVKDRFINFFVGNT
INSSYFPDHPLHSISVRRLKETKDGFMFLTDQSYIDVLPEFRDSYPIKYV
HAFESNNFIYFLTVQRETLDAQTFHTRIIRFCSINSGLHSYMEMPLECIL
TEKRKKRSTKKEVFNILQAAYVSKPGAQLARQIGASLNDDILFGVFAQSK
PDSAEPMDRSAMCAFPIKYVNDFFNKIVNKNNVRCLQHFYGPNHEHCFNR
TLLRNSSGCEARRDEYRTEFTTALQRVDLFMGQFSEVLLTSISTFIKGDL
TIANLGTSEGRFMQVVVSRSGPSTPHVNFLLDSHPVSPEVIVEHTLNQNG
YTLVITGKKITKIPLNGLGCRHFQSCSQCLSAPPFVQCGWCHDKCVRSEE
CLSGTWTQQICLPAIYKVFPNSAPLEGGTRLTICGWDFGFRRNNKFDLKK
TRVLLGNESCTLTLSESTMNTLKCTVGPAMNKHFNMSIIISNGHGTTQYS
TFSYVDPVITSISPKYGPMAGGTLLTLTGNYLNSGNSRHISIGGKTCTLK
SVSNSILECYTPAQTISTEFAVKLKIDLANRETSIFSYREDPIVYEIHPT
KSFISGGSTITGVGKNLNSVSVPRMVINVHEAGRNFTVACQHRSNSEIIC
CTTPSLQQLNLQLPLKTKAFFMLDGILSKYFDLIYVHNPVFKPFEKPVMI
SMGNENVLEIKGNDIDPEAVKGEVLKVGNKSCENIHLHSEAVLCTVPNDL
LKLNSELNIEWKQAISSTVLGKVIVQPDQNFTGLIAGVVSISTALLLLLG
FFLWLKKRKQIKDLGSELVRYDARVHTPHLDRLVSARSVSPTTEMVSNES
VDYRATFPEDQFPNSSQNGSCRQVQYPLTDMSPILTSGDSDISSPLLQNT
VHIDLSALNPELVQAVQHVVIGPSSLIVHFNEVIGRGHFGCVYHGTLLDN
DGKKIHCAVKSLNRITDIGEVSQFLTEGIIMKDFSHPNVLSLLGICLRSE
GSPLVVLPYMKHGDLRNFIRNETHNPTVKDLIGFGLQVAKGMKYLASKKF
VHRDLAARNCIGPLACSSGS.

Where the sequence at the positions 1207-1220 of SEQ ID NO: 3 is ARNCIGPLACSSGS .

The sequence at the positions 1175-1220 of SEQ ID NO: 3 is NPTVKDLIGFGLQVAKGMKYLASKKFVHRDLAARNCIGPLACSSGS.

In a fourth aspect, the disclosure provides a kit for evaluating glioma prognosis, where the kit includes a first primer pair capable of specifically amplifying any cDNA in the first aspect, and/or a first probe capable of specifically hybridizing with any cDNA in the first aspect; and/or the kit includes a second primer pair capable of specifically amplifying any mRNA in the second aspect, and/or a second probe capable of specifically hybridizing with any mRNA in the second aspect; and/or the kit includes an antibody for resisting any protein in the third aspect, and any protein in the third aspect.

Where the first primer pair only needs to be able to specifically amplify any cDNA in the first aspect, and preferably, the first primer pair contains a first primer as shown in SEQ ID NO: 4 and a second primer as shown in SEQ ID NO: 5. The first probe only needs to be able to specifically hybridize with any cDNA in the first aspect, and preferably, the sequence of the first probe contains a sequence as shown in SEQ ID NO: 6. The second primer pair only needs to be able to specifically amplify any mRNA in the second aspect, and preferably, the second primer pair contains a third primer as shown in SEQ ID NO: 7 and a fourth primer as shown in SEQ ID NO: 8. The second probe only needs to be able to specifically hybridize with any mRNA in the second aspect, and preferably, the sequence of the second probe contains a sequence as shown in SEQ ID NO: 9.

Where the sequence as shown in SEQ ID NO: 4 is aatttctgaccgagggaatcat; the sequence as shown in SEQ ID NO: 5 is acgccaaaggaccaatacagtttc; the sequence as shown in SEQ ID NO: 6 is gccaaaggaccaatacagtttctt; the sequence as shown in SEQ ID NO: 7 is taatacgactcactataggggagcacgccaaaggaccaataca; the sequence as shown in SEQ ID NO: 8 is tgaaatatcttgcaagcaaaaagtt; and the sequence as shown in SEQ ID NO: 9 is agcacgccaaaggaccaatacagt.

Where the antibody for resisting any protein in the third aspect can be a monoclonal antibody and/or a polyclonal antibody.

In a fifth aspect, the disclosure provides application of a molecular reagent in preparation of a kit for evaluating glioma prognosis, where the molecular reagent includes at least one of (1)-(8) as follows:

(1) any cDNA in the first aspect; (2) a first primer pair capable of specifically amplifying any cDNA in the first aspect; (3) a first probe capable of specifically hybridizing with any cDNA in the first aspect; (4) any mRNA in the second aspect; (5) a second primer pair capable of specifically amplifying any mRNA in the second aspect; (6) a second probe capable of specifically hybridizing with any mRNA in the second aspect; (7) any protein in the third aspect; and (8) an antibody for resisting any protein in the third aspect.

In a sixth aspect, the disclosure provides a system for evaluating glioma prognosis. The system includes an amplification device, a sequencing device, a computing device and an output device, where the amplification device includes a collection unit and an amplification unit, where the collection unit is used for collecting a template nucleic acid fragment and an amplification primer, and the amplification unit is used for amplifying the template nucleic acid fragment by using the amplification primer to obtain an amplification product; the sequencing device is used for sequencing the nucleic acid sequence of the amplification product to obtain a sequence of the amplification product; the computing device includes a memory and a processor, where a computer program is stored in the memory, and the processor is configured to execute the computer program stored in the memory so as to achieve the following determination: if the sequence of the amplification product contains the sequence of any cDNA in the first aspect and/or the sequence of any mRNA in the second aspect, it is determined that the prognosis of glioma corresponding to the template nucleic acid fragment is poor; and the output device is used for outputting a determination result of the computing device.

In a seventh aspect, the disclosure provides a method for evaluating glioma prognosis, where the method includes a step of detecting whether a to-be-detected glioma sample contains the cDNA and/or the mRNA and/or the protein as described above, and if the to-be-detected glioma sample contains the cDNA and/or the mRNA and/or the protein as described above, it is indicated that the prognosis of a glioma patient corresponding to the to-be-detected glioma sample is poorer. Where the cDNA and/or mRNA can be detected by a method of PCR, NASBA, TMA, RT-RPA, nucleic acid hybridization or high-throughput sequencing, and the protein can be detected by immunoblotting.

The disclosure is further described in detail below by the examples.

### PREPARATION EXAMPLE

This preparation example was used for obtaining glioma samples and obtaining total RNAs and total cDNAs therein.

1211 glioma samples were collected by using operations conforming to the standards of the medical ethical committee, and the pathological characteristics of each glioma sample were determined. Where for each patient from whom a sample was collected, the consent of himself or herself and his or her therapist was obtained before the sample was collected, having a written proof material. Where glioma was diagnosed by utilizing a pathological diagnosis method, the prognosis of the glioma samples was evaluated according to the overall survival of patients corresponding to the glioma samples, and the longer the overall survival of the patients is, the better the prognosis of the glioma is. The characteristics such as the gender, age, pathological grade, tumor category, and overall survival of the patients corresponding to the glioma samples are shown in Table 1.

TABLE 1

| Category | Quantity | |
|---|---|---|
| Total sample number | 1211 | |
| World Health Organization pathological grade | | |
| Grade II | 328 | |
| Grade III | 354 | |
| Grade IV | 529 | |
| Histopathological classification | | |
| Diffuse oligodendroglioma | 110 | |
| Diffuse astrocytoma | 193 | |
| Diffuse oligoastrocytoma | 25 | |
| Anaplastic oligodendroglioma | 99 | |
| Anaplastic oligoastrocytoma | 27 | |
| Anaplastic astrocytoma | 228 | |
| Glioblastoma | 529 | |
| Tumor type | | |
| Primary | 710 | |
| Recurrent | 347 | |
| Secondary | 154 | |
| Age | | |
| Median age | 42 | |
| Distribution | 8-79 | |
| Gender | | |
| Number of males (proportion) | 717 | (59.2%) |
| Overall survival | | |
| Median survival (95% confidence interval) | 863 | (772-1023) |

The total RNAs of the glioma samples were extracted by using a DNA extraction kit (purchased from Qiagen) according to its operation instruction. The total RNAs were detected by an integrity analyzer, and it was confirmed that the RNA integrity number (MN) of the total RNAs was larger than 7.0. Double-stranded cDNA was synthesized by using a reverse transcription kit (purchased from Invitrogen) according to its operation instruction and by taking the total RNAs as a template.

### Example 1

In this example, the cDNAs of the glioma samples synthesized by the preparation example were subjected to PCR verification.

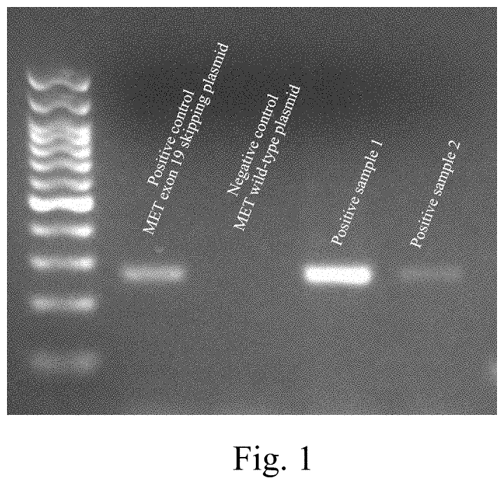

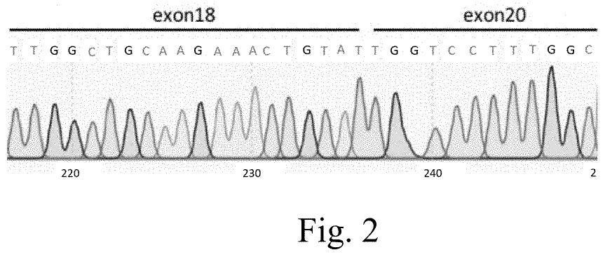

Primers used for the PCR verification were the first primer as shown in SEQ ID NO: 4 and the second primer as shown in SEQ ID NO: 5. The PCR operation was carried out according to the instructions of synthetic primers and the PCR kit. The PCR product was subjected to agarose gel nucleic acid electrophoresis to show whether an amplification band existed or not, as shown in FIG. 1, the appearing amplification bands were recovered by using a DNA gel extraction kit (a QIAquick PCR purification kit, purchased from Qiagen), then Sanger sequencing was performed, and the sequencing result is shown in FIG. 2.

It can be known from FIG. 1 and FIG. 2 that the glioma samples have MET genes with an exon 19 deleted.

### Example 2

In this example, RNAs of the glioma samples collected in the preparation example were sequenced.

An RNA library was constructed from RNA of each sample by using a RNA library construction kit (purchased from Illumina), then RNA sequencing was performed on the RNA library by using a sequencing platform (Illumina HiSeq 2000), and glioma samples containing mRNA as shown in SEQ ID NO: 2 were screened from the RNA sequencing results as shown in Table 2.

TABLE 2

| | | |
|---|---|---|
| | Glioma samples | Glioma samples |
| | with the | with the |
| | sequence of SEQ | sequence of SEQ |
| | ID NO: 2 | ID NO: 2 |
| Category | detected | undetected |

| Quantity | 12 | | 1199 | |
|---|---|---|---|---|
| World Health Organization | | | | |
| pathological grade | | | | |
| II | 0 | | 328 | |
| III | 2 | | 352 | |
| IV | 10 | | 519 | |
| Histopathological classification | | | | |
| Diffuse oligodendroglioma | 0 | | 110 | |
| Diffuse astrocytoma | 0 | | 193 | |
| Diffuse oligoastrocytoma | 0 | | 25 | |
| Anaplastic oligodendroglioma | 0 | | 99 | |
| Anaplastic oligoastrocytoma | 0 | | 27 | |
| Anaplastic astrocytoma | 2 | | 226 | |
| Glioblastoma | 10 | | 519 | |
| Tumor type | | | | |
| Primary | 2 | | 708 | |
| Recurrent | 3 | | 344 | |
| Secondary | 7 | | 147 | |
| Age | | | | |
| Median age | 37 | | 42 | |
| Distribution | 15-53 | | 8-79 | |
| Gender | | | | |
| Number of males (proportion) | 8 | (66.7%) | 709 | (59.1%) |

Overall survival

| | | | | |
|---|---|---|---|---|
| Median survival (95% confidence | 284 | (96-NA) | 866 | (779-1037) |
| interval) | | | | |

It can be seen from Table 2 that 12 glioma samples containing mRNA as shown in SEQ ID NO: 2 were detected with a median survival of 284 days, and 1199 glioma samples not containing the mRNA as shown in SEQ ID NO: 2 were detected, with a median survival of 866 days, which indicated that the prognosis of glioma containing the mRNA as shown in SEQ ID NO: 2 disclosed by the disclosure was poor, so that the mRNA can be used for evaluating glioma prognosis.

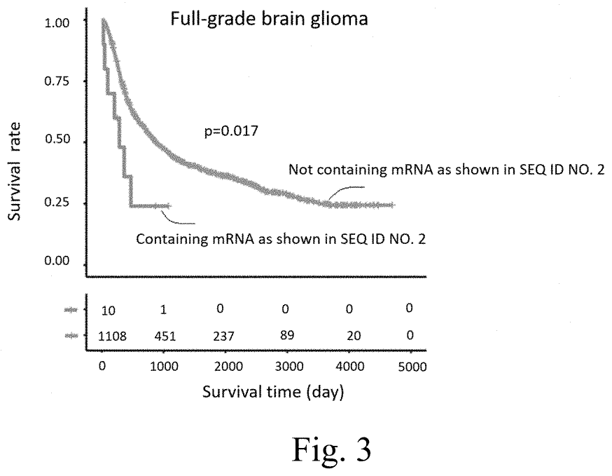

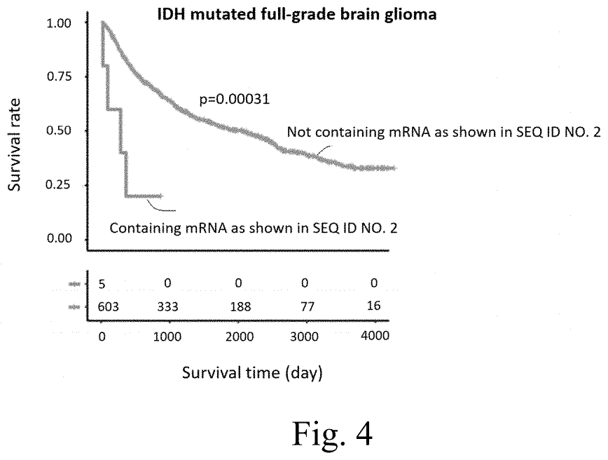

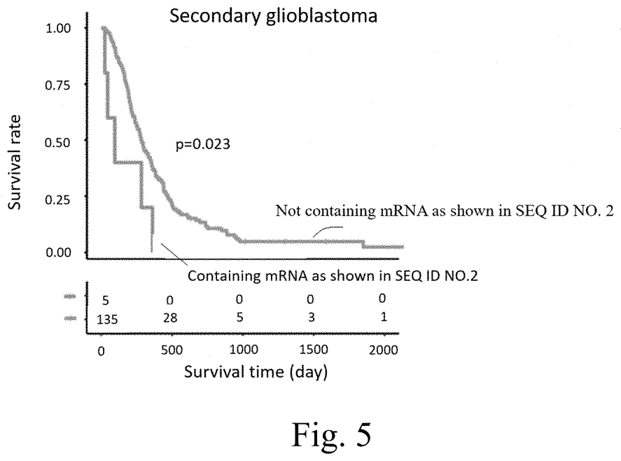

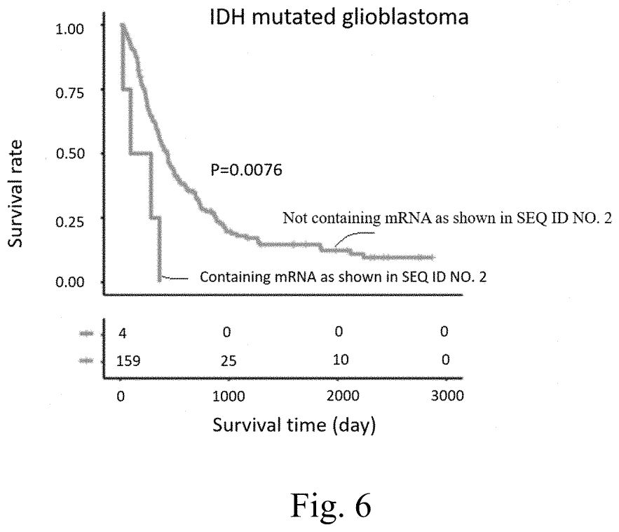

Besides, in the full-grade glioma, the IDH-mutated full-grade glioma, the secondary glioblastoma and the IDH-mutated glioblastoma, the overall survival condition of the glioma samples containing the mRNA shown as the SEQ ID NO: 2 and the overall survival condition of the glioma samples not containing the mRNA shown as the SEQ ID NO: 2 were compared through a survival curve (a Kaplan-Meier curve). Results show that the glioma samples containing the mRNA as shown in SEQ ID NO: 2 were poor in overall survival, as shown in FIGS. 3-6.

The preferred examples of the disclosure are described in detail above in combination with the drawings, however, the disclosure is not limited to the specific details in the above examples, in the technical concept range of the disclosure, the technical solution of the disclosure can be subjected to various simple variations, and these simple variations all belong to the protection range of the disclosure.

In addition, it should be noted that the specific technical features described in the above examples can be combined in any suitable mode without contradiction.

In addition, various different examples of the disclosure can also be combined at will, and as long as the examples do not violate the idea of the disclosure, the examples also should be regarded as the contents disclosed by the disclosure.

1. A cDNA, wherein a sequence of the cDNA contains a fragment of SEQ ID NO: 1, or the sequence of the cDNA contains a complementary fragment of the fragment of SEQ ID NO: 1; wherein the fragment of SEQ ID NO: 1 at least contains a sequence at positions 2635-2652, 2623-2640 or 2627-2645 of SEQ ID NO: 1;
preferably, the fragment of SEQ ID NO: 1 at least contains a sequence at positions 2588-2687 of SEQ ID NO: 1.

2. The cDNA according to claim 1, wherein the cDNA corresponds to a human MET gene, and the sequence of the cDNA does not contain an exon 19 of the human MET gene;
preferably, the cDNA is single-stranded DNA, and the sequence of the cDNA is the sequence as shown in SEQ ID NO: 1, or the sequence of the cDNA is a complementary sequence of the sequence as shown in SEQ ID NO: 1; or,
the cDNA is double-stranded DNA, and the sequence of one single strand of the cDNA is the sequence as shown in SEQ ID NO: 1, and the sequence of the other single strand of the cDNA is the complementary sequence of the sequence as shown in SEQ ID NO: 1.

3. An mRNA, wherein the mRNA is a reverse transcription template for the cDNA according to claim 1.

4. The mRNA according to claim 3, wherein the sequence of the mRNA contains a fragment of SEQ ID NO: 2, wherein the fragment of SEQ ID NO: 2 at least contains a sequence at positions 3820-3837, 3832-3849 or 3827-3845 of SEQ ID NO: 2;
preferably, the fragment of SEQ ID NO: 2 at least contains a sequence at positions 3785-3884 of SEQ ID NO: 2; and
more preferably, the sequence of the mRNA is the sequence as shown in SEQ ID NO: 2.

5. A protein, wherein the protein is encoded by the mRNA according to claim 3.

6. The protein according to claim 5, wherein the amino acid sequence of the protein contains a fragment of SEQ ID NO: 3, wherein the fragment of SEQ ID NO: 3 at least contains a sequence at positions 1207-1220 of SEQ ID NO: 3;
preferably, the fragment of SEQ ID NO: 3 at least contains a sequence at positions 1175-1220 of SEQ ID NO: 3, and
more preferably, the amino acid sequence of the protein is the sequence as shown in SEQ ID NO: 3.

7. A kit for evaluating glioma prognosis, comprising a first primer pair capable of specifically amplifying the cDNA according to claim 1, and/or a first probe capable of specifically hybridizing with the cDNA according to claim 1; and/or,
the kit comprises a second primer pair capable of specifically amplifying an mRNA, wherein the mRNA is a reverse transcription template for the cDNA according to claim 1, and/or a second probe capable of specifically hybridizing with an mRNA, wherein the mRNA is a reverse transcription template for the cDNA according to claim 1; and/or,
the kit comprises an antibody for resisting a protein, wherein the protein is encoded by an mRNA wherein the mRNA is a reverse transcription template for the cDNA according to claim 1, and a protein, wherein the protein is encoded by an mRNA wherein the mRNA is a reverse transcription template for the cDNA according to claim 1.

8. The kit according to claim 7, wherein the first primer pair contains a first primer as shown in SEQ ID NO: 4 and a second primer as shown in SEQ ID NO: 5, and the sequence of the first probe contains a sequence as shown in SEQ ID NO: 6; and
the second primer pair contains a third primer as shown in SEQ ID NO: 7 and a fourth primer as show n in SEQ ID NO: 8, and the sequence of the second probe contains a sequence as shown in SEQ ID NO: 9.

9. Application of a molecular reagent in preparation of a kit for evaluating glioma prognosis, wherein the molecular reagent comprises at least one of (1)-(8) as follows:
(1) the cDNA according to claim 1;
(2) a first primer pair capable of specifically amplifying the cDNA according to claim 1;
(3) a first probe capable of specifically hybridizing with the cDNA according to claim 1;
(4) an mRNA, wherein the mRNA is a reverse transcription template for the cDNA according to claim 1;
(5) a second primer pair capable of specifically amplifying an mRNA, wherein the mRNA is a reverse transcription template for the cDNA according to claim 1;
(6) a second probe capable of specifically hybridizing with an mRNA, wherein the mRNA is a reverse transcription template for the cDNA according to claim 1;
(7) a protein, wherein the protein is encoded by an mRNA wherein the mRNA is a reverse transcription template for the cDNA according to claim 1; and
(8) an antibody for resisting a protein, wherein the protein is encoded by an mRNA wherein the mRNA is a reverse transcription template for the cDNA according to claim 1.

10. A system for evaluating glioma prognosis, comprising an amplification device, a sequencing device, a computing device and an output device; wherein
the amplification device comprises a collection unit and an amplification unit, wherein the collection unit is used for collecting a template nucleic acid fragment and an amplification primer, and the amplification unit is used for amplifying the template nucleic acid fragment by using the amplification primer to obtain an amplification product,
the sequencing device is used for sequencing the nucleic acid sequence of the amplification product to obtain a sequence of the amplification product;
the computing device comprises a memory and a processor, wherein a computer program is stored in the memory, and the processor is configured to execute the computer program stored in the memory so as to achieve the following determination:
in response to determining that the sequence of the amplification product contains the sequence of the cDNA according to claim 1 and/or the sequence of an mRNA, wherein the mRNA is a reverse transcription template for the cDNA according to claim 1, it is determined that the prognosis of glioma corresponding to the template nucleic acid fragment is poor; and
the output device is used for outputting a determination result of the computing device.
